(19) United States
(12) Patent Application Publication
(10) Pub. No.: US 2009/0126048 A1
(43) Pub. Date: May 14, 2009

(54) Method For Growing Forskohlin Containing Coleus In A Temperate Climate

(76) Inventor: Ven Subbiah, Garner, NC (US)

Correspondence Address: PASSE' INTELLECTUAL PROPERTY, LLC, 1717 BRASSFIELD RD., RALEIGH, NC, 27614, US

(21) Appl. No.: 12/267,199

(22) Filed: Nov. 7, 2008

Related U.S. Application Data
(60) Provisional application No. 61/002,297, filed on Nov. 8, 2007.

(51) Int. Cl. A01H 5/00

(52) U.S. Cl. 800/298

(57) ABSTRACT

The present invention is a method of growing and harvesting forskohlin-containing cultivars of the Coleus plant in North America and other geographic areas that results in increased yield of the phytochemical forskohlin per plant, as well as increasing rhizome and root biomass.

### REFERENCE TO RELATED APPLICATIONS

This application is a non-provisional application claiming priority to provisional application 61/002,297 filed Nov. 8, 2007 and incorporated herein by reference in its entirety.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a method of growing and harvesting forskohlin-containing cultivars of Coleus plant in temperate geographic areas such as the temperate areas in North America that results in increased yield of the phytochemical forskohlin per plant, as well as increasing rhizome and root biomass.

2. Description of Related Art

Forskohlin is a naturally occurring chemical well known to be found in the roots of a variety of plants from the Coleus family. Pharmacologically, forskohlin has been demonstrated to increase cellular levels of cyclic AMP (cAMP), and has been thought to provide treatment of various disease states via this mechanism. Many patents have been issued describing forskohlin use for treatment of hypertension, weight reduction, asthma, increased intraocular pressure, and mood disorders. All of these patents, as well as scientific publications, have utilized forskohlin isolated from the roots of various Coleus plants.

The original chemical and the crop have its origin in Asia, and current crops are limited to approximately 50,000 acres in parts of India, Nepal, Thailand, and China. This is primarily because the Coleus plant species does not produce sufficient forskohlin nor sufficient size rhizome or root in temperate zones of the world to be commercially viable. However, a problem with growing Coleus plant species in the tropical and subtropical areas of the world as presently done is that these areas are limited to relatively small acreage, and small scale agricultural practices which limit world-wide supply. Because of the limited areas to support growth of the forskohlin-containing Coleus plants, and limited ability to harvest the forskohlin-containing roots, demand for the chemical has exceeded supply and several groups have attempted to genetically modify plants to produce more forskohlin in the available acreage in the tropical and sub-tropical areas with little commercial success.

### BRIEF SUMMARY OF THE INVENTION

This invention relates to a new method for growing and harvesting Forskohlin-containing cultivars of the Coleus plant, which prior to this invention could only be grown and harvested in select areas of China, India, Thailand, Nepal, and Africa. This new method allows for forskohlin-containing Coleus plants to be grown in temperate arable regions of North and South America, as well as Australia and Europe, thus increasing the supply of forskohlin for a variety of purposes. By planting a seedling in a temperate zone field and then cutting the flowers and inflorescence at least one time during the gowning process, the Coleus plant produces a rhizome and chemical content equal to or greater than Coleus grown in tropical or sub-tropical climates.

A method of growing a forskohlin containing Coleus plant in a temperate climate comprising:
a) planting a forskohlin containing Coleus seedling plant in a temperate climate field;
b) growing the seedling to a mature plant;
c) cutting an inflorescence of the Coleus plant while the Coleus plant is growing such that the flowers are removed from the plant;
d) harvesting the rhizome of the Coleus plant.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

By “coleus plant” refers to any species of the coleus plant which produces forskohlin especially in commercial quantities such as Coleus barbatus. Other known coleus plants include C. amboinicus Lour. Synonym. C. aromaticus Benth; C. parviflorus Benth. Synonym. C. tuberosus Benth.; C. caninus (Roth) Vatke,; C. spicatus Bentham; C. vettiveroides; and C. blumei Benth., Synonym. Plectranthus scutellarioides (L.) R.Br.

By “planting a seedling” refers to the production of a small plant suitable for transplant into an open field in a temperate climate. In one embodiment, under normal greenhouse conditions, Coleus barbatus was grown at a day temperature of 25-28C, and night temperature, 10-20 C with a relative humidity 60-90% until the plant grew to a desired height for transplanting anywhere from about 2-6 inches to a height of 4-6 ft. In one embodiment, the seedling is about 2 to 12 inches in height. In another embodiment, plants grown under standard greenhouse conditions were transferred to loose sandy soil in temperate Eastern North Carolina (in farmland where soy, tobacco, and cotton are also grown), which allows for the tubers to grow with less impediment.

By “planting and growing” as used herein, refers to the planting of the seedling in a temperate farm field and allowed to grow under suitable water conditions and optionally fertilizers pesticides and fungicides sufficient to grow the plant to full size rhizomes. In one actual embodiment, plants were allowed to grow to 6-8 ft, with 50 to 100 branches sprouting in a single plant. Plants demonstrated the capability to withstand one or two frosts grown in this method, however, the roots grow back in the next spring season. If the climatic condition is extremely cold, then multiplication of the plant in greenhouse is applicable. Condition for the green house: Day temp.: 25-30 deg. C. and night temp. 10-20 deg. C. and relative humidity is 80 to 90% is optimal. Forskohlin-containing Coleus plants grown under the above conditions did not require any additional fertilizers or pesticides/fungicides.

By cutting an inflorescence of the Coleus plant while the Coleus plant is growing such that the flowers are removed from the plant, refers to cutting the inflorescence in a manner that allows the plant to continue to grow. Normally leaving a portion of the inflorescence is the easiest if done by machine since hand cutting would be more time consuming but any suitable method could be used as shown in the particular embodiment shown in the example which follows. Many forskohlin-containing Coleus varieties have flowers. In an embodiment, the Coleus barbatus flowered profusely, the inflorescence ranging from 2-3 ft with strong pink flowers. However, the flowers do not set seeds. In addition, both the flower and inflorescence seems to impact the growth and chemical content of the rhizome and roots, reducing the active compound, Forskohlin content. To maximize the forskohlin content, the flowers were chopped at least once and, in one embodiment, 2 or more times during the growth period and prior to rhizome and root harvest. The aerial part of the Coleus barbatus plant was then, in this embodiment, chopped using a tractor, leaving 6 inch aerial stumps.

The rhizome produced by this method can be harvested by any method for harvesting root type crops. An advantage of production in the temperate climates, especially North America, is the ability to mechanically harvest the crop in large farms, therefore, increasing the amount of produced products and doing so at a cheaper cost. In one embodiment, the rhizome was mechanically dug-out using a local peanut harvester and allowed to air dry under sun for two-three days. The air dried materials were collected using sweet potato harvester, where a conveyer belt was applied to collect the rhizome with soil particle and rolling over the conveyer chain was used to remove all soil particles and the clean rhizome were collected into the large baskets and transported to drying.

The rhizome, once harvested, can be processed to extract the forskohlin by any method. For example one extraction method used was as follows:

Root yield per acre: about 10,000 KG fresh weight, dry weight, 1000-1200 KG which was ground to a powder.

1. Extract the powder with Toluene-Methanol (100:1)for more than 6 hours at 55-60 C.

2. Filter and concentrate the fraction to dryness.

3. Take up the extract in small amount of Toluene and extract with Petroleum ether (1:20 ratio).

4. The toluene fraction is enriched forskohlin, the pet. ether picks up all impurities.

5. Repeat the extraction with Pet. ether to enhance the purity to 12, 15 and up to 40% forskohlin content.

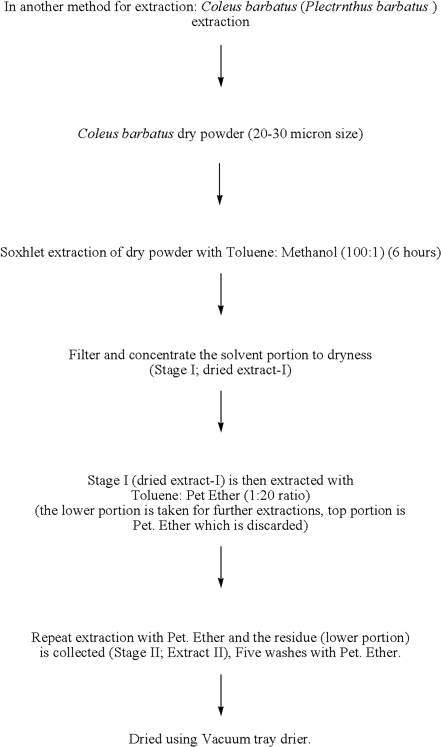

The forskohlin content of the above method of growing and harvesting the coleus plant yields a rhizome with greater than 15% of forskohlin.

### EXAMPLE

Coleus plants were grown in a temperate North Carolina climate according the conditions and methods described above:
1. Life span: It is a perennial plant in wild state, distributed over tropical and subtropical regions. Under cultivation (different varieties like K-8, Garmai, Belgaum etc.,) it requires around 6-8 months for better root growth. It varies depending on the soil conditions.
2. Root settings: Normal roots sets within a month. But tuberous roots starts setting from one and half to two and half months depending on the soil type. E.g.: In red sandy soil it starts around one and half months, but in hard soil type like ours, it takes around two to two and half months after planting. Loose sandy loamy soil will be best for this crop.
3. Determination of the growth: The growth was excellent on the sandy soils on the plains compared to the black loamy soil. The aerial part grew much faster compared to the Asian varieties, and the over all height reached up to 4-5 feet. The inflorescence and flowers started showing up after three months, with robust rhizome and root settings. The mature plants had up to 0.5-1.0 kilogram of the rhizome and roots.
4. Life cycle: Under cultivation, it is around six months in loose soil (red sandy loamy soil), but it takes around eight months in hardy soil for better tuber yield. Once tuber matures, the plant starts shedding of the leaves, this is the indication for harvesting the roots. As it's a perennial plant in wild state, it may take up to one year for tuber harvesting.
5. Over winter: Plant has the capability to withstand winter. Roots survive up to 8 to 10 months However, the plant can sustain 2 or 3 frosts in a North Carolina Climate and then can be replanted accordingly.
6. Frost: Roots may not have the ability to withstand the frost, because this is a heat loving plant and is grown well in day temperatures ranging from 25 to 30 deg. C. and night temperatures of 10-20 deg. C. Relative humidity is between 60 to 70% and an annual rainfall of 100-160 cms. Excess water also results in bacterial wilt and nematodal infestation.
7. Green house: If the climatic condition is extremely cold, then multiplication of the plant in a green house is applicable. Conditions for the green house: Day temp.: 25-30 deg. C. and night temp. 10-20 deg. C. and relative humidity is 60 to 70% is essential, otherwise traditional multiplication through stem cuttings is beneficial.
8. Fertilizers or other conditions to enhance root growth:
   a) Application of NPK at two stages in the ratio of 40 kg: 60 kg: 50 kg. Half of N, whole P and K should be given as basal dose and remaining half of N given after 30 days of planting as top dressing.
   b) Chopping of the flowers one month before harvesting will enhance the root growth.
   c) Create water stress condition one month before harvesting.

The above examples and embodiments are not intended to be limiting. Changes in Coleus plants exact growing conditions, trimming of the flowers and their timing are all within the skill in the art in view of this patent application. The claims which follow are intended, therefore, to be read in a broad manner in view of the substitutions and teachings herein.

What is claimed is:

1. A method of growing a forskohlin containing Coleus plant in a temperate climate comprising:
a) planting a forskohlin containing Coleus seedling plant in a temperate climate field;
b) growing the seedling to a mature plant;
c) cutting an inflorescence of the Coleus plant while the Coleus plant is growing such that the flowers are removed from the plant;
d) harvesting the rhizome of the Coleus plant.

2. A method according to claim 1 wherein step c) is repeated at least once with a subsequent flowering inflorescence in the Coleus plant.

3. A method according to claim 1 wherein the Coleus plant is selected from the group consisting of Coleus barbatus.

4. A method according to claim 1 wherein the rhizome is processed to remove the forskohlin from the rhizome.

5. A Coleus rhizome produced according to the method of claim 1.
